(19) United States
(12) Patent Application Publication
(10) Pub. No.: US 2015/0104802 A1
(43) Pub. Date: Apr. 16, 2015

(54) SYSTEMS AND METHODS FOR DEVELOPING QUANTIFIABLE MATERIAL STANDARDS FOR FEEDSTOCKS AND PRODUCTS USED IN ADDITIVE MANUFACTRUING PROCESSES

(71) Applicants: Paul Reep, Ojai, CA (US); Kabir Sagoo, Ojai, CA (US)

(72) Inventors: Paul Reep, Ojai, CA (US); Kabir Sagoo, Ojai, CA (US)

(21) Appl. No.: 14/529,036

(22) Filed: Oct. 30, 2014

Related U.S. Application Data
(60) Provisional application No. 61/889,681, filed on Oct. 11, 2013.

(51) Int. Cl. C12Q 1/68

(52) U.S. Cl. CPC C12Q 1/686
USPC 435/6.12; 73/866

(57) ABSTRACT

Methods and systems for enabling additive manufacturing feedstock materials to communicate with supervisory control and data acquisition (SCADA) systems by way of molecular markers. Markers can be DNA encoded with information or other small molecules that emit a biochemical, chemical, fluorescent, or conductive signal; markers also include additives that transmit empirical data about resistivity, density, weight, volumetric information or other properties of the 3D printed object. The methods to inject materials with markers are to use various solvents or integration as a dry formulation, blended by weight, and concentration. Markers can be applied to feedstock materials entirely, or integrated at various locations on a 3D-printed object while it is being printed or as a thin layer while the object undergoes post-processing treatment.

### CROSS-REFERENCE TO RELATED APPLICATIONS

This application claims the benefit of U.S. Provisional Patent Application No. 61/889,681 which was filed on Nov. 4, 2013.

### BACKGROUND

Additive manufacturing refers to the industrial technologies for ‘printing’ objects layer-by-layer; this type of manufacturing is colloquially referred to as ‘3D printing’. Additive manufacturing relies on a computer and 3D modeling software to produce a parsed and layered model of the object to be printed. Data is input into the additive manufacturing printer using specific software to lay down or add successive layers of liquid, powder, sheet material or other feedstock, in a layer-upon-layer fashion to fabricate the 3D object. The feedstock for additive manufacturing systems may be dispensed by several different methods such as, extrusion deposition, wire deposition, granular deposition, powder-bed, inkjet-head deposition, lamination, and photopolymerization. The terms ‘feedstock’ or ‘materials’ apply to powders, polymeric materials, metals, wires, ceramics, adhesives, and other materials used as raw materials for additive manufacturing.

Tighter process control and traceability are imperative to improving the state of the industry's quality control and standardization techniques. Currently most additive manufacturing processes operate in ‘open loop’ with little to no data passed back to the device on feedstock quality or refinements needed during the operation.

### SUMMARY OF THE INVENTION

The present invention relates to the fields of additive manufacturing, materials, synthetic biology, biochemistry, and microbiology. The invention adds multiple layers of security, origination information and communication capability to dry materials, aqueous materials, powders, billet, extrusions, castings, wire including coated and/or lubricated, resins, and blends used as raw materials for additive manufacturing feedstocks. The invention allows for the establishment of a new series of quality standards and technologies to improve the manufacturing process, quality of the final product, aid in automation, feedstock characterization and solve problems with blended feedstocks by creating standardization of a new a line of manufacturing machinery, parts, protocols, tooling, software, feedstocks, taggants, and sensor networks.

The present invention further relates to systems and methods for identifying, measuring and controlling key parameters of additive manufacturing by developing processes to provide feedback to the efficiency and efficacy of such processes, while standardizing the identification of underlying feedstock to facilitate and enhance large-scale use of additive manufacturing technologies. The present invention further relates to a system and method of embedding data to quantifiably determine origination and pedigree of feedstocks and products using highly specified molecular or physical markers or ‘taggants’, and to create secondary structured taggants within or on the surface of the manufactured object that communicate with additive manufacturing sensors, software and data acquisition network (SCADA).

In particular, the invention allows additive manufacturing systems to intelligently obtain information on the feedstock used in the fabrication process by identifying key taggants present in the feedstock through a system of sensors and software. The taggant contains information on the feedstock specification and chain of custody of the material used in the additive manufacturing process similar identification protocols on product containers or shipping labels. The taggants may include such ‘pedigree’ information comprising, but not limited to, alloy composition, thermal characteristics, concentration, viscosity, hardness, tensile strength, particle size, particle distribution, particle shape, specific surface, interparticle friction as measured by packing and flow characterization, equipment used in preparation of the feedstock, melting point, density, chemical formula, state of charge, vendor or manufacturer, point of origin, date of manufacture, identification number, special conditions or exceptions to specification, and presence of binders or other additives.

The taggant may also contain information on the method the feedstock should be used by additive manufacturing comprising injection or operational volume, speed, temperature, pressure, and flow rate. The process control variables can be controlled with better precision when multiple feedstocks are blended together or multiple different feedstocks are used to create an object.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 shows a method of additive manufacturing; and

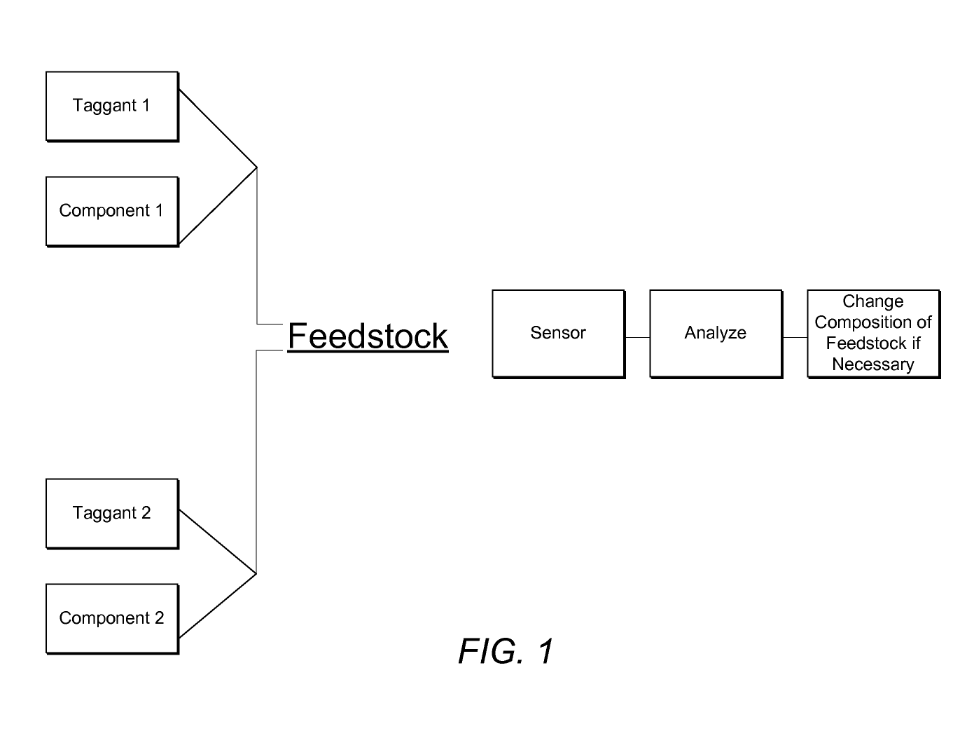

FIG. 2 shows a method of determining characteristics of a product.

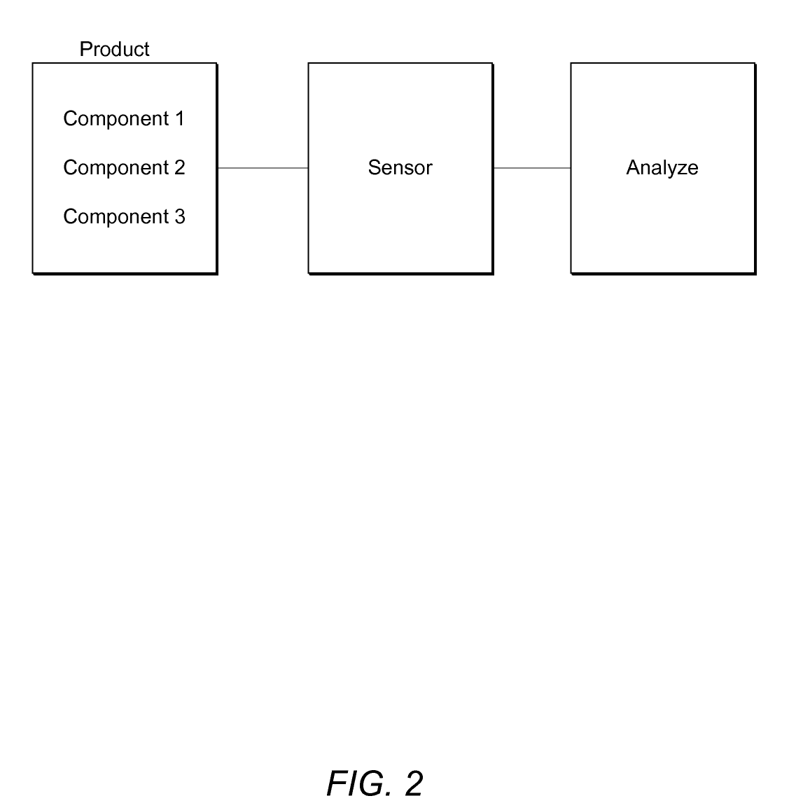

### DETAILED DESCRIPTION OF THE PREFERRED EMBODIMENTS

Specific taggants may be selected depending on the end use or manufacturing method chosen to implement the additive manufacturing process. For example, taggants of moly-niobium and tungsten rhenium are good alloys for applications up to 3000 C, DNA markers may be good for polymer material use at temperatures up to 200 C, while Silica balloons could be selected as good optical markers, due to their well-characterized structural properties.

There may be an additional necessity to identify and add taggant to mark process fluids, such as water, solvents, lubricants, coatings or catalysts used in the additive manufacturing process. This data on additional agents would be used to validate a formulation or gain information on if process was performed correctly, for example, ensuring proper lubrication, addition of a solvent or catalyst, and what quantity of material was added. Taggants can also be used and introduced to the additive manufacturing system by dedicated vessel, spray, mist, cuvette or well.

The data contained in molecular markers or by the presence of the taggants can be used to avoid possible negative interactions between feedstocks or optimize feedstock usage during the manufacturing process. Data is limited on feedstock blending formulations with unknown and untested chemistries, instability could occur due changes in the material. During the manufactured object's lifecycle, volatility and instability may occur through physical and chemical changes to the object, as the object wears or is exposed to the atmosphere, including expansion and contraction, denaturing, and oxidation, and therefore reducing the object's reliability or function. There is a critical need for the feedstocks to communicate data about these life cycle processes during testing and production back to the additive manufacturing system, or any system capable of reading the taggants in the feedstock such as a handheld device to ensure quality and guarantee performance specifications. The handheld device or other such sensor in the SCADA network may rely on CT scans or ultrasonics in some embodiments.

The use of taggants may allow for easier regulatory compliance when additive manufacturing products are required by regulators to have disposal protocols following the ultimate use of the product. This system may also aid in the permitting a facility for additive manufacturing use due to the increase robustness associated with material handling protocols.

The use of taggants may also allow for changes to the physical and chemical properties of the object created by the additive manufacturing process, in different states. In one example the object's material durability could be improved or retarded. The object's physical structure may contain a series of pores or voids that could have taggant inserted into the pores or in other embodiments the taggants could be deposited during the manufacturing process. The taggants could be used to increase or decrease, rigidity, hardness, flexion or other physical properties to the material. The taggants, and their placement, can be proprietary in their location assignment, and can create a secondary structure, such as a 2D ‘X’ in 3D space, to determine point of origin, ensure quality, and add additional information to the object. By placing the taggant in a specific manner and creating a secondary structure, it would be possible to aid in the post inspection and quality check process by ensuring the specified structure is present and functioning correctly. In one embodiment the taggant could contain conductive material and create an implanted circuit that could be measured for a known conductivity or resistivity to determine if a quality standard was met.

The additive manufacturing systems described in the invention will have the ability to sense, detect, measure, or quantify, empirical attributes about the feedstocks, enabling these sensor-equipped process control and supervisory control and data acquisition (SCADA) systems to adjust operational parameters of the additive manufacturing process, from information contained in the feedstock. This approach not only aids in traceability and determining point of origin, but also makes the additive manufacturing process far more efficient and precise. Information the SCADA system and computer automated measurement and control systems (CAMAC) gathers from the taggants may be used alone and in conjunction with other materials to calibrate the process control system and the associated sensors.

Some embodiments of the present invention also relate to a system and method, which utilizes DNA and other molecular taggants are used to gather information and store additional information in various feedstocks and objects. Some embodiments of the invention maybe used to give feedstocks and objects manufactured additively a way to communicate with 3D printers or other readers information about their origins and prevent the proliferation of counterfeit, low quality or defective products.

In certain implementations various feedstock producers may identify, measure and control key parameters in relation to specific feedstocks to maximize the efficiency and efficacy of such additive manufacturing processes while standardizing the underlying parameters to facilitate and enhance large-scale manufacturing.

Feedstocks and materials may come from original equipment manufacturers (OEM), materials producers, additive service bureaus, research institutions, or other materials providers in the advanced manufacturing supply chain, which will need to be blended in correct ratios to ensure a uniform, and quality product is delivered to downstream customers. As different products are refined along the supply chain, the invention postulates that specifically identified taggants are added to specified steps to identify the mixture. These taggants are added in pre-determined concentrations, which can be readily identified and added with by software or manual control. In one embodiment of the invention DNA taggants are made of synthetic sequences of nucleic acid or by isolating natural occurring sequences of nucleic acid from yeast, human cell lines, bacteria, animals, plants and the like. In other embodiments the isolated nucleic acid is from extremophilic organisms. The length of the DNA taggant usually ranges from 100 bases to about 10 kilo bases, preferably 1 kb to 3 kb in length. In specific embodiments the DNA maybe encapsulated in a proactive coating or polymer to increase the DNAs resistance to environmental degradation and/or damage.

Quantification of the DNA taggant may be accomplished by using quantitative polymerase chain reaction (qPCR). By using this method in combination with High Resolution Melt (HMR) analysis the entire sequence of DNA does not need to be sequenced as the difference between the known sequence and the target sequence can be compared as melting curve changes.

Due to DNA large coding capacity a small sequence of DNA can contain a large amount of information, which can be used to embed data directly throughout the product, not necessarily limited to a region or single ‘tag’. DNA taggants can also be used to authenticate items or products in a supply chain and further add to the track and traceability of material after leaving the manufacturing facility. As indicated above, when producers may wish to blend several different feedstocks together, such as a resin and a metal, different taggants or combinations of taggants can be added to each component. A downstream customer can then test the product and check against a database stored locally or in a networked configuration to know the product's origin, specifications, and performance criteria. The protection provided by this system is layered and robust because points of authentication are available on multiple levels. Suppliers and buyers of 3D printed products or raw materials can check for the presences or absence of a taggant, the concentration of that taggant, the state of the taggant being activated, inactivated, protected or unprotected, and the oligonucleotide sequence of the taggant. Electrostatic taggants can also be added, where the state of charge can be altered or induced on a dry feedstock or end product, to help in the detection by the SCADA network. This technique may also help to more discretely control processing in the distribution and manufacturing, leading up to integration with the additive manufacturing system.

Additional taggants could comprise, a luminescent taggant, a phosphorescent taggant, a chemoluminescent taggant, a fluoroluminescent taggant, an optical or machine readable taggant, a nano-particle taggant, a mirco-spheare taggant, an electromagnetic taggant, a probe insertion for surrogate authentication of the DNA, a X-ray probe, a CT probe, a chemical taggant having a visible, infra-red, near infra-red and ultra-violet absorber and reflector component chemistry, a taggant that is reusable, a color-shifting ink taggant, a pigment taggant, a catalyst taggant, a taggant that has antigenic reaction for instant, non-forensic assay, and a taggants that emit sound on activation.

In another embodiment the taggant may be able to be activated by laser or other electromagnetic waves to provide a signal back to the user. The activated taggant can then be recognized and interpreted by the user with a corresponding reader device.

Additional taggant detected and authenticated methods could comprise utilizing, Optical Microscope, Scanning Electron Microscope (SEM), Transmission Electron

Microscope (TEM), Field Ion Microscope (FIM), Scanning Tunneling Microscope (STM), Atomic Force Microscope (AFM), X-ray diffraction topography (XRT), Energy-Dispersive X-ray spectroscopy (EDX), Wavelength Dispersive X-ray spectroscopy (WDX),X-Ray Diffraction (XRD), Mass spectrometry, Impulse excitation technique (IET), Secondary Ion Mass Spectrometry (SIMS), Electron Energy Loss Spectroscopy (EELS), Auger electron spectroscopy, X-ray photoelectron spectroscopy (XPS), ICP-MS : Inductively Coupled Plasma Mass Spectroscopy (ICP-MS), Ultraviolet-visible spectroscopy (UV-vis), and Capillary flow porometry (CFP).

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A method for monitoring the characteristics of a feedstock comprising:
a) introducing a quantity of least one component of a taggant into feedstock;
b) passing the feedstock by a sensor configured to identity and measure taggants;
c) analyzing the measured quantity of taggants to determine characteristics of the feedstock; and
d) altering the quantity of a taggant containing component to achieve a desired feedstock characteristic.

2. A method as recited in claim 1, wherein the step of analyzing further comprises comparing the identified taggant to a table to determine when and where a component was manufactured.

3. A method as recited in claim 1, wherein the step of analyzing further comprises identifying and measuring taggants in components to validate a formulation of feedstock.

4. A method as recited in claim 1, wherein the step of analyzing further comprises identifying and measuring taggants in components of a feedstock to prevent negative interactions of components.

5. A method for detecting the characteristics of a product of additive manufacturing comprising:
a) introducing a quantity of a taggant with a known life cycle into at least one component of a feedstock used to manufacture a product;
b) sensing the taggant within a completed product to determine characteristics of the product.

6. A method as recited in claim 5, wherein the step of sensing comprises comparing sensed taggant information and comparing it to a table to determine the current life cycle status of a product.

7. A method as recited in claim 6, wherein the step of sensing comprises comparing the sensed taggant data to a table to determine the appropriate disposal protocol to use.

8. A method as recited in claim 6, wherein the step of sensing comprises comparing the sensed taggant data to a table to determine changes in the properties of a taggant to adjust operational parameters.

9. A method as recited in claim 6, wherein the step of sensing comprises comparing sensed taggant data to determine changes in the properties of the material.
